(19) United States
(12) Patent Application Publication
(10) Pub. No.: US 2026/0334813 A1
(43) Pub. Date: Oct. 29, 2026

(54) METHOD FOR ACQUIRING THICKNESS OF PATIENT AND X-RAY IMAGING SYSTEM

(71) Applicant: GE Precision Healthcare LLC, Waukesha, WI (US)

(72) Inventors: Yi Xu, Beijing (CN); Jiabao Zhang, Beijing (CN); Enquan Huang, Beijing (CN); Ruoxi Zhang, Beijing (CN); Buer Qi, Beijing (CN)

(21) Appl. No.: 19/660,503

(22) Filed: Apr. 28, 2026

(30) Foreign Application Priority Data
Apr. 28, 2025 (CN) 202510543750.1

(51) Int. Cl. A61B 5/107; A61B 5/00

(52) U.S. Cl. CPC A61B 5/1072; A61B 5/0077

(57) ABSTRACT

The present application provides a method for acquiring a thickness of a patient and an X-ray imaging system. The method for acquiring a thickness of a patient includes: acquiring optical image data and depth image data captured by a camera; generating a point cloud on the basis of the optical image data and the depth image data, where the point cloud can be displayed in one or a plurality of views; extracting a target region in the point cloud, the target region including a region of interest of the patient; and acquiring a thickness value of the patient on the basis of a thickness of at least one key region divided within the target region, or acquiring a thickness value of the patient on the basis of a distance between a first surface and a second surface of the patient that are fitted.

### CROSS-REFERENCE TO RELATED APPLICATIONS

This application is based on and claims priority to Chinese Patent Application No. 202510543750.1, filed on Apr. 28, 2025, the entire contents of which is herein incorporated by reference.

### TECHNICAL FIELD

The present invention relates to medical imaging technology, and more specifically, to a method for acquiring a thickness of a patient, an X-ray imaging system, and a non-transitory computer-readable storage medium.

### BACKGROUND

In an X-ray imaging system, radiation from an X-ray source is emitted toward a subject, and the patient is usually a patient in a medical diagnosis application. Some of the radiation passes through the patient and impacts a detector, which is divided into a matrix of discrete elements (e.g., pixels). The detector elements are read to generate an output signal on the basis of the amount or intensity of radiation that impacts each pixel region. The signal can then be processed to generate a medical image that can be displayed for review, and the medical image can be displayed in a display apparatus of the X-ray imaging system.

Generally, the exposure protocol is usually selected according to the size of the patient. The dose required for exposure is usually larger for patients of larger sizes and smaller for patients of smaller sizes. Currently, in some cases, the exposure protocol may be selected on the basis of one or more of the age, gender, weight, height, and the like of the patient, which is not accurate enough for the selection of the exposure protocol. This may cause the patient to receive an excessive dose, or may cause the patient to receive insufficient dose to obtain a prepared image.

### SUMMARY OF THE INVENTION

The present invention provides a method for acquiring a thickness of a patient, an X-ray imaging system, and a non-transitory computer-readable storage medium.

An exemplary embodiment of the present invention provides a method for acquiring a thickness of a patient, including acquiring optical image data and depth image data captured by a camera; generating a point cloud on the basis of the optical image data and the depth image data, where the point cloud can be displayed in one or a plurality of views; extracting a target region in the point cloud, the target region including a region of interest of the patient; and acquiring a thickness value of the patient on the basis of a thickness of at least one key region divided within the target region, or acquiring a thickness value of the patient on the basis of identifying a distance between a first surface and a second surface of the patient.

An exemplary embodiment of the present invention further provides an X-ray imaging method, including: acquiring optical image data and depth image data captured by a camera; generating a point cloud on the basis of the optical image data and the depth image data, where the point cloud is capable of being displayed in one or a plurality of views; extracting a target region in the point cloud, the target region including a region of interest of the patient; acquiring a thickness value of the patient on the basis of a thickness of at least one key region divided within the target region, or acquiring a thickness value of the patient on the basis of identifying a distance between a first surface and a second surface of the patient; and selecting exposure parameters on the basis of the thickness value to carry out exposure on the patient.

An exemplary embodiment of the present invention further provides an X-ray imaging system, including a control unit capable of executing the method for acquiring a thickness of a patient.

An exemplary embodiment of the present invention further provides an X-ray imaging system, including an apparatus for acquiring a thickness of a patient. The apparatus for acquiring a thickness of a patient includes an acquisition module, a generation module, an extraction module, and a calculation module. The acquisition module is configured to acquire optical image data and depth image data captured by a camera. The generation module is configured to generate a point cloud on the basis of the optical image data and the depth image data, where the point cloud can be displayed in one or a plurality of views. The extraction module is configured to extract a target region in the point cloud. The target region includes a region of interest of the patient. The calculation module is configured to acquire a thickness value of the patient on the basis of a thickness of at least one key region divided within the target region, or acquire a thickness value of the patient on the basis of identifying a distance between a first surface and a second surface of the patient.

An exemplary embodiment of the present invention further provides a non-transitory computer-readable storage medium, configured to store a computer program, which when executed by a computer, causes the computer to execute the method for acquiring a thickness of a patient.

Other features and aspects will become apparent from the following detailed description, accompanying drawings, and claims.

### BRIEF DESCRIPTION OF THE DRAWINGS

The present invention can be better understood by means of the description of the exemplary embodiments of the present invention in conjunction with the drawings, in which:

FIG. 1 is a schematic diagram of an X-ray imaging system according to some embodiments of the present invention;

FIG. 2 is a schematic diagram of a control logic of a control unit in an X-ray imaging system according to some embodiments of the present invention;

FIG. 3 shows output results of a classification learning model according to some embodiments of the present invention;

FIG. 4 is a schematic diagram of a first surface and a second surface of a patient that are fitted according to some embodiments of the present invention;

FIG. 5 is a flowchart of a method for acquiring a thickness of a patient according to some embodiments of the present invention;

FIG. 6 is a specific flowchart of generating a point cloud in the method for acquiring a thickness of a patient shown in FIG. 5;

FIG. 7 is a flowchart of a method for acquiring a thickness of a patient according to some embodiments of the present invention; and

FIG. 8 is a flowchart of a method for acquiring a thickness of a patient according to some other embodiments of the present invention.

### DETAILED DESCRIPTION

Specific implementations of the present invention will be described below. It should be noted that in the specific description of said implementations, for the sake of brevity and conciseness, the present description cannot describe all of the features of the actual implementations in detail. It should be understood that in the actual implementation process of any implementation, just as in the process of any one engineering project or design project, a variety of specific decisions are often made to achieve specific goals of the developer and to meet system-related or business-related constraints, which may also vary from one implementation to another. Furthermore, it should also be understood that although efforts made in such development processes may be complex and tedious, for those of ordinary skill in the art related to the content disclosed in the present invention, some design, manufacture, or production changes made on the basis of the technical content disclosed in the present disclosure are only common technical means, and should not be construed as the content of the present disclosure being insufficient.

Unless defined otherwise, technical terms or scientific terms used in the claims and description should have the usual meanings that are understood by those of ordinary skill in the technical field to which the present invention belongs. The terms “first” and “second” and similar terms used in the description and claims of the patent application of the present invention do not denote any order, quantity, or importance, but are merely intended to distinguish between different constituents. The terms “one” or “a/an” and similar terms do not express a limitation of quantity, but rather that at least one is present. The terms “include” or “comprise” and similar words indicate that an element or object preceding the terms “include” or “comprise” encompasses elements or objects and equivalent elements thereof listed after the terms “include” or “comprise”, and do not exclude other elements or objects. The terms “connect” or “link” and similar words are not limited to physical or mechanical connections, and are not limited to direct or indirect connections.

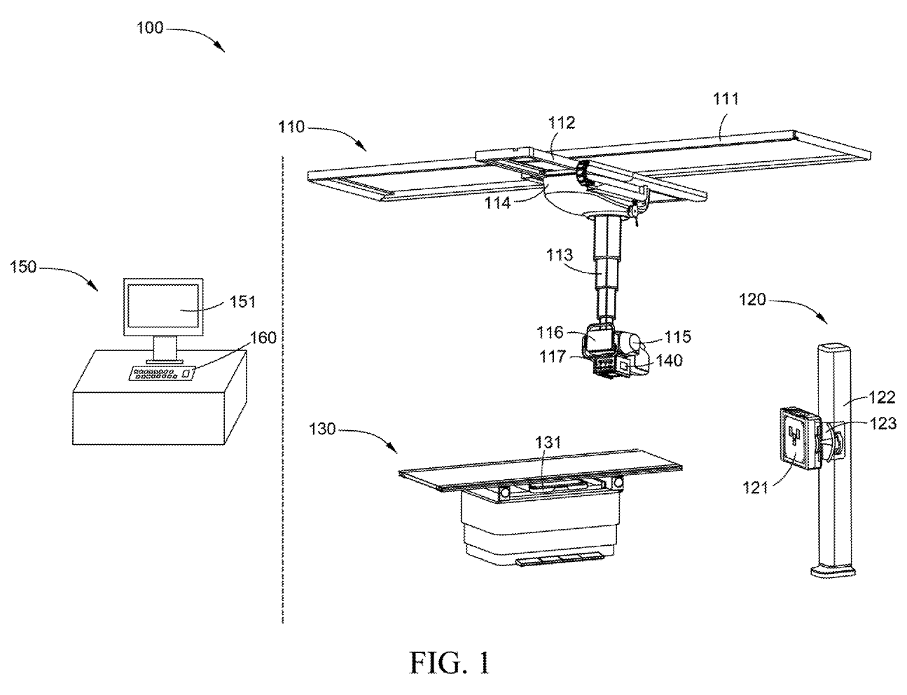

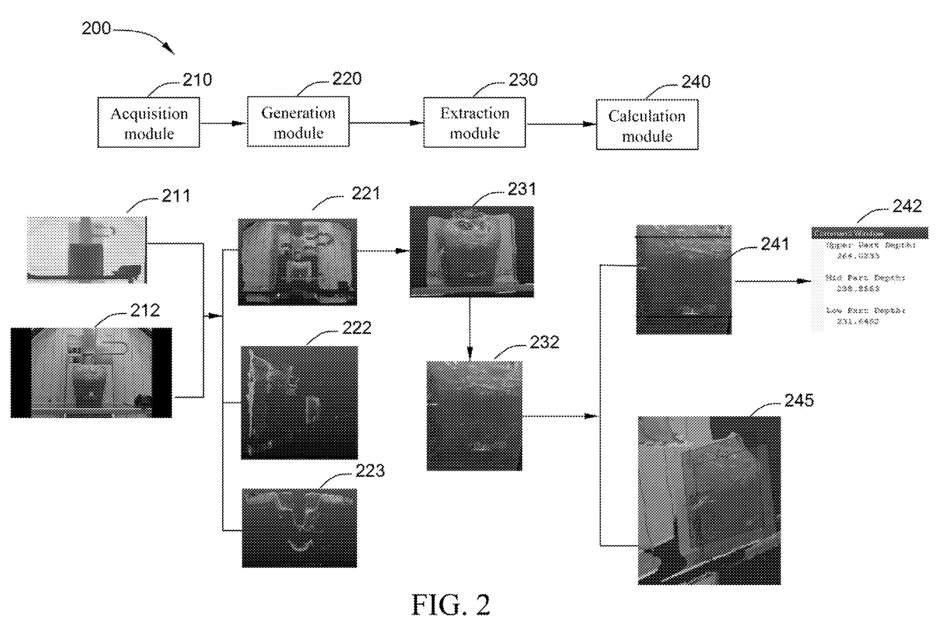

FIG. 1 shows an X-ray imaging system 100 according to some embodiments of the present invention, and FIG. 2 shows a schematic view of a collimator of the X-ray imaging system as shown in FIG. 1. As shown in FIG. 1, the X-ray imaging system 100 includes a suspension apparatus OTS 110, a wall stand apparatus 120, and an examination table apparatus 130. The suspension apparatus OTS 110 includes a longitudinal guide rail 111, a transverse guide rail 112, a telescopic cylinder 113, a sliding member 114, a tube assembly 115, and an OTS console 116.

For ease of description, in the present application, the x-axis, y-axis, and z-axis are defined as the x-axis and y-axis being located in the horizontal plane and perpendicular to one another, and the z-axis being perpendicular to the horizontal plane. Specifically, the direction in which a longitudinal guide rail 111 is located is defined as the x-axis, the direction in which a transverse guide rail 112 is located is defined as the y-axis direction, and the direction of extension of the telescopic cylinder 113 is defined as the z-axis direction, and the z-axis direction is the vertical direction.

The longitudinal guide rail 111 and the transverse guide rail 112 are perpendicularly arranged, the longitudinal guide rail 111 being mounted on a ceiling and the transverse guide rail 112 being mounted on the longitudinal guide rail 111. The telescopic cylinder 113 is configured to carry the tube assembly 115. The sliding member 114 is provided between the transverse guide rail 112 and the telescopic cylinder 113. The sliding member 114 may include components such as a rotating shaft, a motor, and a reel. The motor can drive the reel to rotate around the rotating shaft, which in turn drives the telescopic cylinder 113 to move along the z-axis and/or slide relative to the transverse guide rail. The sliding member 114 is capable of sliding relative to the transverse guide rail 112, i.e., the sliding member 114 is capable of driving the telescopic cylinder 113 and/or the tube assembly 115 to move in the y-axis direction. Further, the transverse guide rail 112 can slide relative to the longitudinal guide rail 111, which in turn drives the telescopic cylinder 113 and/or the tube assembly 115 to move in the x-axis direction.

The telescopic cylinder 113 includes a plurality of cylinders having different inner diameters, and the plurality of cylinders can be sleeved, sequentially from bottom to top, in the cylinder located thereabove, thereby achieving telescoping, and the telescopic cylinder 113 can be telescopic (or movable) in the vertical direction, i.e., the telescopic cylinder 113 can drive the tube assembly 115 to move along the z-axis direction. The lower end of the telescopic cylinder 113 is further provided with a rotating part, and the rotating part can drive the tube assembly 115 to rotate. The size of an aperture of the collimator 117 dictates the irradiation range of X-rays, namely the size of the region of an exposure field of view (FOV). X-rays can pass through the aperture of the collimator to a region of interest (ROI) of the patient, and other X-rays are absorbed by the shutters to prevent the patient from absorbing an excess unnecessary dose. The position of the X-ray source and the collimator 117 in the transverse direction determines the position of the exposure field of view (FOV) on the patient.

The OTS console 116 is mounted on the tube assembly 115. The OTS console 116 includes user interfaces such as a display screen and a control button so as to be configured to perform pre-capturing preparations, such as patient selection, protocol selection, positioning, etc.

The wall stand apparatus 120 includes a first detector 121, a wall stand 122, and a connecting member 123. The connecting member 123 includes a support arm that is vertically connected in the height direction of the wall stand 122 and a rotating bracket that is mounted on the support arm, and the first detector 121 is mounted on the rotating bracket. The wall stand apparatus 120 further includes a detector driving apparatus that is arranged between the rotating bracket and the first detector 121, which is driven by the detector driving apparatus to move in a direction parallel to the height direction of the wall stand 122 in the plane held by the rotating bracket, and the first detector 121 can further be rotated relative to the support arm to form an angle with the wall stand. The first detector 121 has a plate-like structure whose orientation is variable so that the X-ray incident surface can become vertical or horizontal depending on the incident direction of the X-rays.

A second detector 131 is included on the examination table apparatus 130, and the selection or use of the first detector 121 and the second detector 131 may be determined on the basis of a captured site of patient and/or a capture protocol, or may be determined on the basis of the position of the patient that is obtained from a camera capture, so as to conduct a supine, prone or standing capture examination. FIG. 1 only shows an example diagram of a wall stand and an examination table, and it should be understood by those skilled in the art that wall stands and/or examination tables of any form or arrangement may be selected, or only the wall stand may be mounted, and the wall stand and/or examination table is not intended to limit the overall solution of the present application.

During the imaging process, the center of the X-ray source, i.e., the central ray of the X-ray beam, often needs to be aligned with the center of the detector. In some embodiments, the X-ray source and the detector can be controlled in a linked manner, for example, the position and angle of the X-ray source can be controlled by controlling the movement of the suspension apparatus, and then, by means of monitoring information of the X-ray source that is provided on the detector, the detector can be automatically moved or rotated to a position that is aligned with the X-ray source, i.e., a linked control of the two can be achieved. Vice versa, the detector can be controlled to move or rotate and then the X-ray source can be automatically moved or rotated, on the basis of monitoring information provided on the X-ray source, to a position that is aligned with the detector.

In some embodiments, the X-ray imaging system 100 further includes a camera unit 140, and the camera unit 140 is aligned with the detector so as to be configured to acquire a real-time image of the patient. In addition, the camera is able to acquire an image of the detector, etc. The real-time image can be used to acquire at least one of the thickness, height, position, body position, posture, etc. of the patient.

Specifically, the camera unit 140 is mounted on the suspension apparatus 110, and further, on the side of the collimator 117. In other embodiments, the camera unit 140 may also be fixedly mounted at any position in a scan room, for example, mounted on a wall, and further, mounted on a wall of the detector aligned with the wall stand. Of course, a plurality of camera units may also be mounted in the scan room, that is, they may be fixedly mounted, or mounted on a movable support, or mounted on the X-ray imaging system, as long as they can assist the user in positioning and protocol selection.

The camera unit may include one or more cameras, for example, a digital camera, an analog camera, etc., or a depth camera, an infrared camera, or an ultraviolet camera, etc., or a 3D camera, a 3D scanner, etc., or a red, green, and blue (RGB) sensor, an RGB depth (RGB-D) sensor, or other devices that can capture color image data of a target subject. “RGB” stands for three color channels: red, green, and blue, and “D” stands for depth channel. In some embodiments, the camera unit 140 is further provided with a control module that can control the rotation of the camera unit to adjust the capture range of the camera unit. In other embodiments, the camera unit is a panoramic camera that can capture an image of the entire body of the patient. In some embodiments, the optical image acquired by the camera unit is not limited to a single optical image, but may also include a dynamic real-time video stream, i.e., a series of real-time optical images.

In some embodiments, the camera unit is capable of acquiring RGB optical images and depth images. In this implementation, the camera image not only records color information of a scene, but also includes depth or distance information of each pixel relative to the camera.

The RGB color information refers to color data of each pixel point in the camera image, and is represented by different intensity combinations of the red, green, and blue color channels. The RGB color information enables the visible light camera to capture color details in the scene, thereby generating a color image. The depth information records a physical distance between each pixel in the scene and the visible light camera. In the RGB-D camera, the depth information may be obtained by a variety of techniques, such as structured light, Time of Flight (ToF), or stereo vision. Of course, there may also be an infrared (IR) sensor to improve the ranging performance, so as to improve the accuracy of depth data acquisition. A depth map may be represented in the form of a grayscale map in which each pixel value corresponds to an actual distance, rather than brightness or color information.

The X-ray imaging system 100 further includes a display unit 150. Specifically, the display unit 150 can include any form of display screen, which may be a main display screen that is located in the control room, a display screen of the OTS console 116 that is located in the scan room, or a mobile display, such as a tablet computer, a mobile phone, etc. The display unit 150 is operatively connected to the camera unit. The display unit 150 includes a user interface 151, and the user interface 151 is configured to display a real-time optical image, a depth image, and the like, and of course, may also display any view of the generated point cloud or a calculated thickness value of the patient.

The X-ray imaging system further includes an input unit 160, configured to receive a user operation. The input unit 160 can include an input device such as a touchscreen, a keyboard, a mouse, a voice-activated control unit, or any other suitable input device, and a user can input an operation signal/control signal into the control unit by means of the input unit 160.

The X-ray imaging system 100 further includes a control unit (not shown in the figures), which may be a main control unit that is located in the control room, an OTS control unit that is mounted on the suspension apparatus, a mobile or portable control unit, or any combination of the above. The control unit may include a source control unit and a detector control unit. The source control unit is configured to command the X-ray source to emit X-rays for image exposure. The detector control unit is configured to select a suitable detector among a plurality of detectors, and to coordinate the control of various detector functions, such as automatically selecting a corresponding detector on the basis of the position or posture of the patient. Alternatively, the detector controller may perform various signal processing and filtering functions, specifically, for initial adjustment of a dynamic range, interleaving of digital image data, and the like. In some embodiments, the control unit may provide power and timing signals for controlling the operation of the X-ray source and the detector.

In some embodiments, the control unit may also be configured to use a digitized signal to reconstruct one or more required images and/or determine useful diagnostic information corresponding to the patient. The control unit may include one or more dedicated processors, graphics processing units, digital signal processors, microcomputers, microcontrollers, application-specific integrated circuits (ASICs), field-programmable gate arrays (FPGAs), or other appropriate processing apparatuses.

Of course, the X-ray imaging system may also include other numbers or configurations or forms of control units, for example, the control unit may be local (e.g., co-located with one or more X-ray imaging systems 100, e.g., within the same facility and/or the same local network). In other implementations, the control unit may be remote and thus only accessible by means of a remote connection (for example, by means of the Internet or other available remote access technologies). In a specific implementation, the control unit may also be configured in a manner similar to the configuration of cloud technology, and may be accessed and/or used in a manner substantially similar to the manner of accessing and using other cloud-based systems.

Generally, in the exposure control of the X-ray imaging system, for example, in the process of determining the exposure parameters, it is usually necessary to acquire the thickness of the patient to determine the exposure dose on the basis of the thickness. According to the previous method for acquiring a thickness of a patient, the thickness of the patient is typically estimated directly on the basis of the depth image acquired by the camera unit. Specifically, the thickness of the patient is directly acquired on the basis of the depth information in the region of interest of the patient, or the thickness of the patient may be estimated on the basis of the transformation relationship, for example, between the angle at which the camera unit is mounted and the image source distance. The disadvantage brought by such estimation is that when there is a large distance between the patient and the detector, the estimated thickness value will have a large error, which will also cause a large deviation in the subsequent selection of the exposure dose. Therefore, how to accurately acquire the thickness of the patient is important for the exposure control of the X-ray imaging system.

On the basis of the above mentioned problems, the present application provides a method for acquiring a thickness value by using plane fitting or by acquiring, on the basis of a target region divided in a point cloud obtained on the basis of an RGB image and a depth image acquired by a camera and on the basis of at least one key region related to the captured site in the target region, an average value of the thickness in the key region, so as to accurately obtain the thickness of the patient, so that the thickness is no longer dependent on the distance between the patient and the detector.

Specifically, FIG. 2 shows a schematic diagram of an apparatus 200 for acquiring a thickness of a patient according to the present application. As shown in FIG. 2, the apparatus 200 for acquiring a thickness of a patient includes an acquisition module 210, a generation module 220, an extraction module 230, and a calculation module 240. The acquisition module 210 is configured to acquire optical image data 212 and depth image data 211 captured by a camera. The generation module 220 is configured to generate a point cloud on the basis of the optical image data 212 and the depth image data 211. The point cloud can be displayed in one or a plurality of views. The extraction module 230 is configured to extract a target region in the point cloud. The target region includes at least a region of interest of a patient. The calculation module 250 is configured to acquire a thickness value of the patient on the basis of a thickness of at least one key region divided within the target region or on the basis of a distance between a first surface and a second surface of the patient that are fitted.

Specifically, the acquisition module 210 can be connected to a camera unit (e.g., the camera unit 140 shown in FIG. 1) of the X-ray imaging system, to acquire the real-time optical image 212 and the depth image 211.

The generation module 220 is further configured to perform registration on the basis of the optical image data 212 and the depth image data 211 and generate an initial point cloud, and post-process the initial point cloud to obtain the point cloud.

A point cloud is a data set including a large number of points, and each point has its specific coordinates (x, y, z) in a three-dimensional space. These points are typically used to represent the three-dimensional shape and features of an object or environment.

In some embodiments, due to the inconsistent size of the optical image data and the depth image data, the optical image data and the depth image data usually need registration first, for example, rigid registration, and of course, non-rigid registration may also be used, so that the optical image data and the depth image data have the same size. Then, an initial point cloud without color elements is acquired on the basis of the registered depth image, and then the optical image data is applied to the initial point cloud, so that the initial point cloud has color elements (i.e., corresponding to the color of the optical image data). Of course, the method is not limited to the steps mentioned above, and may also be any other method suitable for generating a point cloud on the basis of an optical image and a depth image.

Specifically, the post-processing includes hole filling and downsampling. The hole filling may fill blank pixel points generated by the 2D to 3D transformation, and the downsampling may reduce the data amount of the image and reduce the image processing cost of subsequent steps.

In the above process of generating the initial point cloud, due to the 2D to 3D spatial transformation, many pixel points will have no pixel value, and at this time, it is necessary to perform hole filling on the initial point cloud to fill the blank points caused by the spatial transformation. Of course, the hole filling may be performed using any existing method or filling rule. For example, the pixel value of the adjacent pixel point on the left side of the blank pixel point may be used for filling, or the maximum of the pixel values in the adjacent pixel points may be used for filling, or the minimum of the pixel values in the adjacent pixel points may be used for filling. Of course, any other suitable filling rule may also be used.

After the hole filling is completed, the initial point cloud may be further downsampled, so that the number of points can be reduced, the data can be easier to process and analyze, and the efficiency of subsequent image processing can be improved. Any suitable method or rule may be used to perform downsampling, for example, different downsampling factors may be set. For example, adjacent pixels or signal points may be averaged to reduce the resolution of data, or the maximum in each region may be selected as the representative of the region. Specifically, different methods may be selected on the basis of the number of point clouds. Alternatively, the downsampling factor may also be selected or customized by the user. For example, the factor may be displayed on a user interface and selected by the user, or may be set according to the parameters of the camera set in the X-ray imaging system, or may be selected according to the capture type of the user. For example, for the capture type of the physical examination, there is no need for particularly accurate image quality, so the downsampling factor may be set to be larger, so as to reduce the data amount as much as possible.

In some embodiments, since the point cloud is 3D, one or a plurality of views of the point cloud may be presented to the user. For example, a front view 221, a side view 222, and a top view 223 may be presented to the user, and of course, any other view required by the user may also be presented. The presented views may be completely selected and switched by the user. For example, the 3D point cloud is presented to the user, and the user may select any view to be displayed on the 3D point cloud. The at least one view is further used in subsequent plane fitting, which will be described in detail later.

Since the camera image captured by the camera generally has a large angle of view and includes not only the region of interest of the patient and the detector region, but also a background part, such as the environment around the detector and the environment of the scan room, after the point cloud is generated, the point cloud also includes the background part and the imaging part, and the extraction module 230 is further required to extract the target region in the point cloud, so that the extracted target region only includes the region of interest of the patient as much as possible.

Specifically, the extraction module 230 is further configured to remove a background part in the point cloud to obtain the target region 231. Specifically, the removing the background part in the point cloud includes identifying a classification map corresponding to the point cloud on the basis of a classification learning model, and extracting the target region on the basis of the classification map or removing the background part on the basis of a source image distance (SID). Specifically, after removing the background part, the extraction module 230 further includes separating the detector region and retaining only the region of interest of the patient, as shown in the image 232.

Specifically, the point cloud includes a relatively large range of background environment, so in order to calculate the thickness more accurately, the background environment needs to be removed first. In some embodiments, the background part may be extracted and removed on the basis of the source image distance (SID). Specifically, in an example where the SID is 1.8 m, that is, the distance between the X-ray source and the detector is 1.8 m, the distance between the X-ray source and the patient is definitely less than 1.8 m, and the distance between the X-ray source and the background environment is definitely far more than 1.8 m. The distance and angle between the camera and the X-ray source are fixed depending on the mounting position of the camera, so the background part may be separated on the basis of a method in which the thickness in the point cloud exceeds a threshold, which depends on the image source distance (SID). Therefore, the environment background part can be conveniently removed on the basis of the SID.

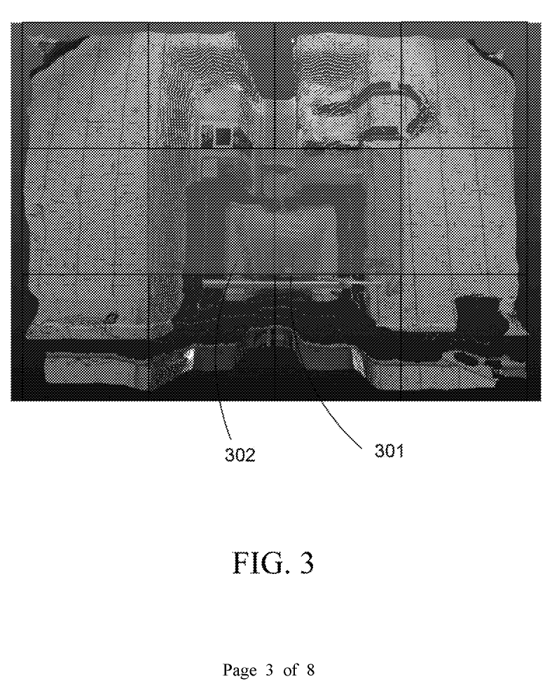

In some other embodiments, the classification map corresponding to the point cloud may be identified on the basis of a classification learning model, and the target region is extracted on the basis of the classification map. Specifically, the learning model may be a classification model. For example, the depths in the point cloud may be classified. Specifically, the depths of the point cloud may be classified according to the size (43*43) of the exposure field of view (FOV). The depths may be classified into 2 types, 3 types or more types. The point cloud may be input into the classification learning model to output a corresponding classification map, which may show a classification level corresponding to each block with the size of FOV, and then the target region may be extracted on the basis of the classification map. Specifically, FIG. 3 shows output results of a classification learning model according to some embodiments of the present invention. As shown in FIG. 3, the front view in the entire point cloud is divided into blocks of 3 rows and 4 columns according to the size of the FOV, and a thickness value of each block is classified respectively to output its corresponding level. FIG. 3 shows two blocks with high confidence, namely, blocks 301 and 302, that is, the region of interest of the patient is located in blocks 301 and 302 to extract the imaging part. Of course, in order to further divide the imaging part, blocks 301 and 302 may also be divided into smaller blocks which are then input into the classification model, and the region of interest of the patient is acquired by continuous iteration. Of course, the point cloud may not be divided on the basis of the FOV initially, and the image may be divided on the basis of any other suitable size.

In still other embodiments, the environment background part may be removed in any other suitable manner, as long as the region of interest can be retained.

Referring back to FIG. 2, after the background part is removed, the image 231 includes the region of interest of the patient and the detector region. In order to further separate the detector region and retain only the region of interest 232 of the patient, in some embodiments, the extraction module 230 further segments the region of interest of the patient on the basis of a histogram of the imaging part in the point cloud. In other embodiments, the extraction module 230 further segments the region of interest of the patient on the basis of a difference or contrast between a clothes color of the patient and a detector color. In still other embodiments, the extraction module 230 may further segment the region of interest of the patient on the basis of an identified boundary of the patient or an identified anatomical structure of the patient. Of course, the region of interest of the patient may also be extracted on the basis of any other suitable segmentation or extraction method, so that the target region only includes the region of interest of the patient.

Although the above shows the method of separating the background part first and then segmenting the detector region, those skilled in the art should understand that these two steps can be completely combined, for example, the region of interest of the patient may be directly segmented from the point cloud on the basis of the histogram, and the like.

Although the above shows that the point cloud is generated first and then the target region is extracted, those skilled in the art should understand that it is also possible to perform registration on the basis of the optical image data and the depth image data first before extracting the target region, which includes the region of interest of the patient and the detector region, and then generate the point cloud on the basis of the target region of the optical image data and the target region in the depth image data, that is, the generated point cloud also includes only the region of interest and the detector region. Then, the point cloud image including only the region of interest is identified on the basis of the above-described method.

In some embodiments, the calculation module 240 can divide the target region into at least one key region 241 on the basis of a captured site of the patient, calculate an average value of a thickness value in the at least one key region respectively, and calculate, when the target region is divided into a plurality of key regions, a thickness value of the patient in each key region respectively, and calculate the overall thickness value 242 of the patient on the basis of a weight of each key region.

Specifically, the calculation module 240 can be connected to a control unit or a processor of an exposure protocol of the X-ray imaging system, so as to receive an exposure protocol of the patient. The exposure protocol includes information such as the captured site, body position, age, weight, and medical history of the patient. The calculation module 240 is further configured to divide the key region on the basis of the captured site of the patient.

Specifically, when there are a plurality of key regions, a thickness value of each key region is calculated respectively. The thickness value may be the average value or greatest value of thickness in the key region. After the thickness of each key region is acquired, the overall thickness of the patient may be calculated on the basis of the weight of each key region, and of course, the average value or greatest value thickness of each key region may also be used as an overall thickness value of the patient.

Specifically, in an example of taking chest X-rays, the primary requirement is to calculate or acquire the thickness of the chest, while avoiding the calculation of the thickness of the neck. If the thickness of the neck is added to or considered in the calculation of the final thickness of the patient, the thickness of the chest will be underestimated, so the target region can be divided into one or a plurality of key regions, for example, three key regions, on the basis of the captured site. The first region is the shoulders, the second region is the lungs in the middle, and the third region is the abdomen. Then, the thickness of each key region is calculated respectively. Of course, when the captured part is the spine, the key regions should be the vertebrae and the back, and the key regions may also be divided according to the definition. For each captured site, a corresponding key site and a position of the key site in the image may be predefined, and the definition may be stored in the X-ray imaging system in the form of a lookup table or a database.

In some embodiments, identify a specific anatomical structure, in some embodiments, it is usually required to move the detector panel to be aligned with the captured site of the patient, which will also make the position of the captured site in the optical image relatively fixed, so in the target region extracted from the point cloud, the positions of different anatomical structures in the target region are also relatively fixed. For example, from top to bottom, one-fifth to two-fifths of the target region can cover the shoulders, two-fifths to four-fifths of the target region can completely cover the lungs, and the remaining one-fifth is the abdomen, so the key regions can be divided on the basis of the positions of the anatomical structures in the target region.

In some other embodiments, the anatomical structure of the patient may also be identified according to an image recognition or learning model, and the required anatomical structure is selected according to the captured site to determine the key region. Of course, the thickness calculated by this method will be more accurate, and the calculation amount required will be larger.

The above method of calculating the thickness by dividing the key regions is relatively simple and the process is also simple. However, the patient and the detector cannot be completely separated in the thickness direction (the emission direction of the X-rays), especially when there is a certain interval between the patient and the detector, the calculated thickness of the patient will include the distance between the patient and the detector in addition to the thickness of the patient, so the calculated thickness has a large error. Therefore, the present application further provides calculation of the thickness of the patient on the basis of two planes (as shown by 245 in FIG. 2) of the patient obtained by plane fitting.

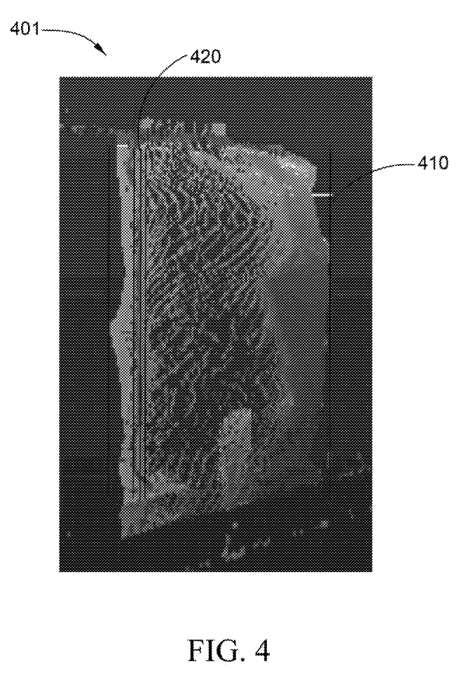

Specifically, FIG. 4 shows a simple schematic diagram of a first surface and a second surface of a patient that are fitted according to some embodiments of the present invention. As shown in FIG. 4, the calculation module 240 can fit the first surface and the second surface of the patient on the basis of a side view or a top view of the point cloud. Although FIG. 4 shows a side view of the point cloud, of course, the fitting can also be performed on the basis of the top view of the point cloud. Of course, any other suitable view can also be used.

As shown in FIG. 4, the calculation module 240 can identify the first surface 410 of the patient away from the detector on the basis of a thickness value in the target region, fit and identify the second surface 420 of the patient close to the detector, and acquire a thickness value of the patient on the basis of the distance between the first surface 410 and the second surface 420. The first surface may be the front chest or the back, and the second surface is the other one of the front chest or the back. Specifically, for a PA view, the back of the patient is the first surface, and the chest of the patient is the second surface.

Specifically, it is a relatively common and conventional practice to identify the first surface 410 away from the detector. For example, the first surface of the patient may be identified on the basis of the greatest value of the thickness in the target region. Specifically, since thickness values in the extracted target region are all known, by determining the position or number of layers where the greatest thickness value is located, the layer where the greatest thickness value is located (perpendicular to the emission direction of the X-rays) is defined as the first surface of the patient. Of course, the plane of the patient away from the detector may also be defined by any other suitable manner, for example, by identifying the boundary or by mounting or providing a camera on the side surface (perpendicular to the surface of the detector).

After acquiring the first surface 410 of the patient, it is most important to acquire the second surface of the patient. Specifically, a plurality of planes of the patient close to the detector may be fitted, the plurality of planes including the surface of the detector, and then the second surface is identified in the plurality of planes on the basis of continuity of a histogram of the target region. Since the second surface of the patient is close to the detector, even if there is a certain interval, the detector and the second surface of the patient cannot be easily identified due to the deformation of the spatial transformation in the point cloud, so a plurality of planes may be fitted at the part close to the detector, the plurality of planes including two surfaces of the detector and the plane where the patient is located, and then the second surface of the patient may be identified on the basis of the continuity of the histogram. Of course, the surface of the detector may also be removed from the plurality of planes on the basis of the thickness of the detector, and then the surface closest to the detector is defined as the second surface.

Specifically, the histogram of the point cloud can describe local features of the point cloud data. Specifically, the histogram of the point cloud corresponding to the patient is a curve with a peak value, and the histogram corresponding to the detector is a straight line with a constant value. Therefore, on the basis of the difference between the histograms corresponding to different elements, the boundary between the detector and the patient can be segmented on the basis of the continuity of the histogram, and then the second surface of the patient can be accurately identified.

After the two surfaces of the patient are acquired, the thickness value of the patient may be acquired on the basis of the distance between the two surfaces. Of course, the two surfaces of the patient are not necessarily parallel, and when the two surfaces are not parallel, the thickness value of the patient may be obtained on the basis of a greatest value or average value of the distance between the two surfaces.

In some embodiments, the above-mentioned apparatus for acquiring a thickness of a patient is performed by a plurality of different modules respectively. However, those skilled in the art should understand that the above-mentioned plurality of modules may also be integrated together, or the above-mentioned functions may be directly performed by the control unit.

In some embodiments, after the X-ray imaging system acquires the thickness of the patient by using the above-mentioned apparatus for acquiring a thickness of a patient or the control unit, the X-ray imaging system may control the exposure dose according to the calculated thickness, and then perform exposure.

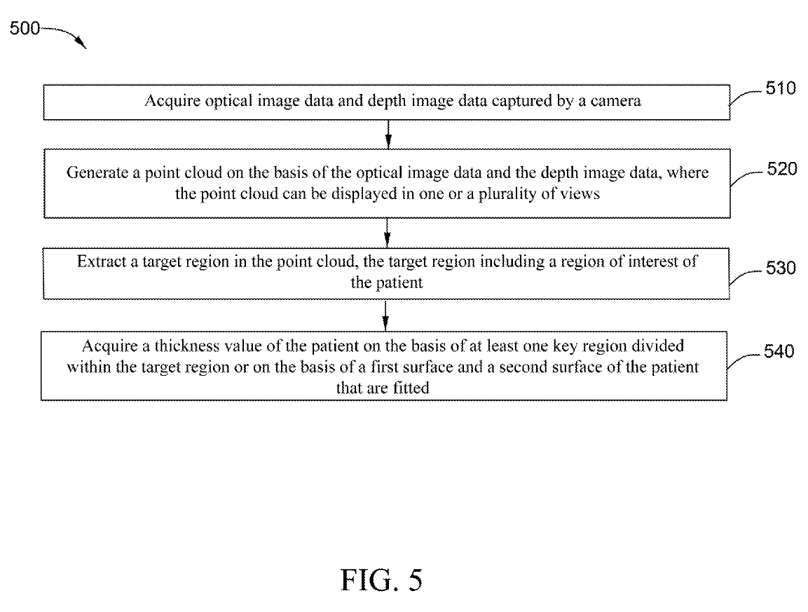

FIG. 5 shows a flowchart of a method 500 for acquiring a thickness of a patient according to some embodiments of the present invention. As shown in FIG. 5, the method 500 for acquiring a thickness of a patient includes step 510, step 520, step 530, and step 540.

In step 510, optical image data and depth image data captured by a camera are acquired.

In step 520, a point cloud is generated on the basis of the optical image data and the depth image data, where the point cloud can be displayed in one or a plurality of views.

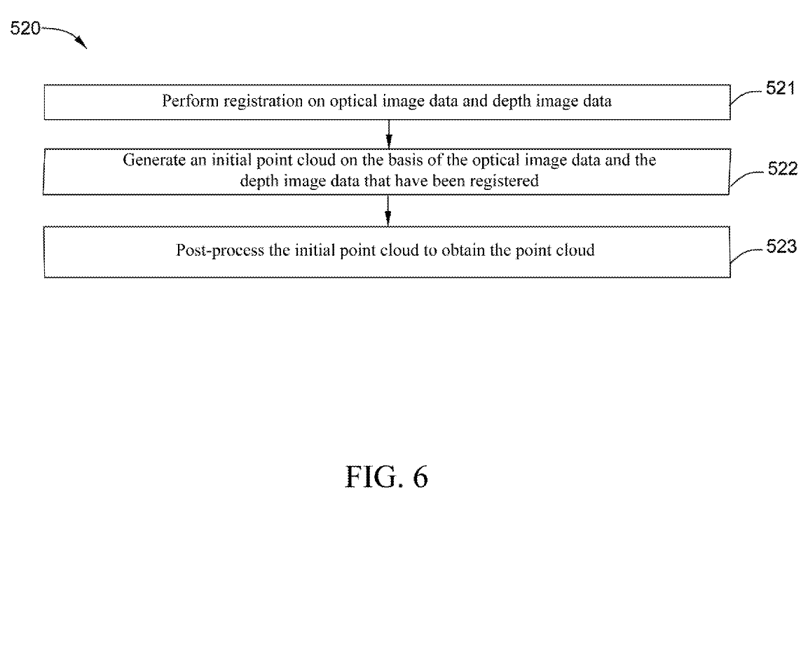

Specifically, FIG. 6 shows a flowchart of generating a point cloud 520 in the method for acquiring a thickness of a patient shown in FIG. 5. As shown in FIG. 6, step 520 further includes step 521, step 522, and step 523.

In step 521, registration is performed on the optical image data and the depth image data. Specifically, the optical image data and the depth image data may be registered in a rigid or non-rigid manner, so that the optical image data and the depth image data have the same size.

In step 522, an initial point cloud is generated on the basis of the optical image data and the depth image data that are registered. Specifically, an initial point cloud without color elements is acquired on the basis of the registered depth image, and then the optical image data is applied to the initial point cloud, so that the initial point cloud has color elements.

In step 523, the initial point cloud is post-processed to obtain the point cloud. Specifically, the post-processing includes hole filling and downsampling. The hole filling may fill blank pixel points generated by the 2D to 3D transformation, and the downsampling may reduce the data amount of the image and reduce the image processing cost of subsequent steps.

For the point cloud acquired on the basis of steps 521 to 523, because the point cloud is 3D, one or a plurality of views of the point cloud may be presented to the user through a graphical user interface (for example, the display interface 151 or 116 shown in FIG. 1), and the one or the plurality of views include a front view, a side view, a top view, and the like.

In step 530, a target region in the point cloud is extracted. The target region includes a region of interest of the patient.

Specifically, the extracting a target region in the point cloud includes: removing a background part in the point cloud to obtain the target region including the region of interest of the patient.

More specifically, in some embodiments, the removing a background part in the point cloud includes identifying a classification map corresponding to the point cloud on the basis of a classification learning model, and extracting the target region on the basis of the classification map. Specifically, the target region may be divided into a plurality of blocks according to a preset size (for example, the size of the exposure field of view (FOV)), each block is input into the classification learning model, the classification level of a thickness value in the block is output, one or more blocks with higher confidence are obtained, and then the one or more blocks are further divided and input into the classification learning model until the region of interest of the patient is obtained.

In some other embodiments, the removing a background part in the point cloud includes removing the background part on the basis of a source image distance (SID). Specifically, the distance between the X-ray source and the patient is definitely less than the source image distance, and the distance between the X-ray source and the background environment is definitely far more than the source image distance. The distance and angle between the camera and the X-ray source are fixed, so the background part can be separated on the basis of a method in which the thickness in the point cloud exceeds a threshold.

In some other embodiments, the environment background part in the point cloud may be removed in any other suitable manner, as long as the region of interest can be retained.

After the background part of the point cloud is removed, the detector region may be removed on the basis of a histogram of the imaging part, a difference or contrast between the clothes color of the patient and the detector color, an identified boundary of the patient, or an identified anatomical result of the patient, so that the target region includes only the region of interest of the patient.

Of course, before the point cloud is generated, the target region may be divided from the depth image and the optical image, and then the point cloud is generated on the basis of the target region of the optical image data and the target region in the depth image data.

In step 540, a thickness value of the patient is acquired on the basis of at least one key region divided within the target region or on the basis of a first surface and a second surface of the patient that are fitted.

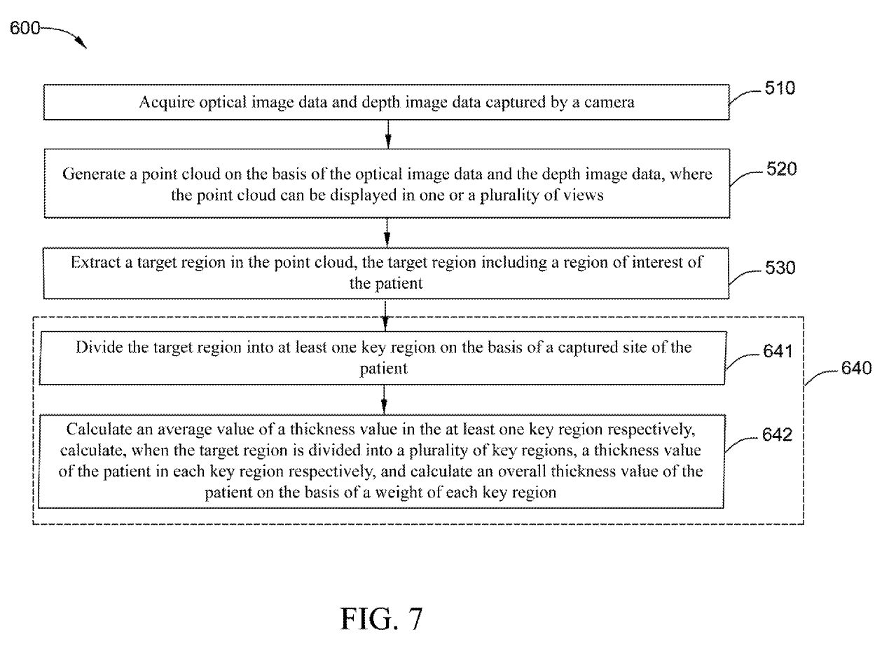

In some embodiments, FIG. 7 shows a flowchart of a method 600 for acquiring a thickness of a patient according to some embodiments of the present invention. As shown in FIG. 7, step 640 of acquiring the thickness of the patient may include step 641 and step 642.

In step 641, the target region is divided into at least one key region on the basis of a captured site of the patient. In some embodiments, the exposure protocol of the patient includes information such as the captured site, body position, age, weight, and medical history of the patient, and one or a plurality of key regions may be obtained on the basis of the captured site of the patient. For example, in an example of taking chest X-rays, the target region may be divided into three key regions: shoulders, lungs, and abdomen. The neck is excluded from the key regions. When calculating a thickness value, the thickness of the neck is not included. Specifically, the key region may be determined on the basis of the position of the anatomical structure in the target region, or the key region may be determined on the basis of the identified anatomical result of the patient.

In step 642, an average value of a thickness value in the at least one key region is calculated respectively, and when the target region is divided into a plurality of key regions, a thickness value of the patient in each key region is calculated respectively, and an overall thickness value of the patient is calculated on the bas is of a weight of each key region. Of course, an average or greatest value of thickness of each key region may also be used as the overall thickness value of the patient.

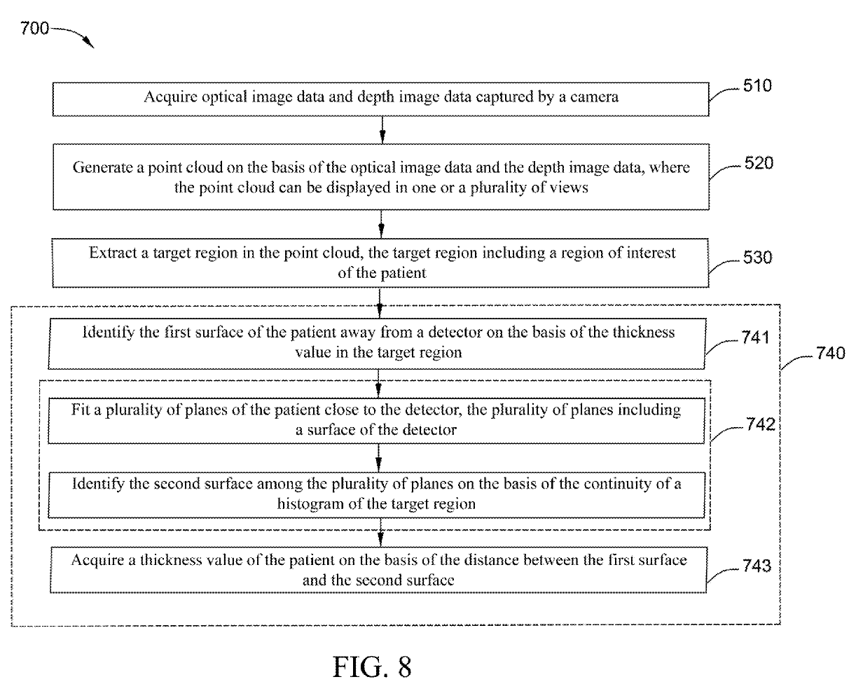

In some other embodiments, FIG. 8 shows a flowchart of a method 700 for acquiring a thickness of a patient according to some embodiments of the present invention. As shown in FIG. 8, step 740 of acquiring the thickness of the patient includes step 741, step 742, and step 743.

In step 741, the first surface of the patient away from a detector is identified on the basis of a thickness value in the target region. Specifically, for a PA view, the back of the patient is the first surface, and the chest of the patient is the second surface. Specifically, the first surface and the second surface of the patient may be identified on the basis of the point cloud or the side view or the top view of the target region in the point cloud.

Specifically, a position at which the greatest value of thickness in the target region is located is determined, and a surface on which the position is located is defined as the first surface of the patient. Specifically, by determining the position or number of layers where the greatest thickness value in the target region is located, the layer where the greatest thickness value is located (perpendicular to the emission direction of the X-rays or the thickness direction) is defined as the first surface of the patient.

In step 742, the second surface of the patient close to the detector is fitted and identified.

Specifically, step 742 further includes: fitting a plurality of planes of the patient close to the detector, the plurality of planes including the surface of the detector, and identifying the second surface in the plurality of planes on the basis of continuity of a histogram of the target region.

In step 743, a thickness value of the patient is acquired on the basis of the distance between the first surface and the second surface.

In some embodiments, for the thickness of the patient acquired by the above method, the system may control the exposure dose according to the calculated thickness, and then perform exposure.

In some embodiments, in addition to the two methods for calculating the thickness on the basis of the target region, the two methods may also be used in combination. For example, the key region is first divided into one or a plurality of key regions, then the detector region is completely separated in the thickness direction (i.e., the emission direction of the X-rays) on the basis of plane fitting, and then a thickness value of each key region is calculated. For another example, in addition to the camera mounted on the side of the collimator, a movable camera is also mounted on the side perpendicular to the surface of the detector. When the patient stands in front of the detector, the thickness of the patient can be calculated on the basis of the camera on the side, and in this case, there is no interference such as the detector in the thickness direction of the patient, so the calculated thickness is extremely accurate. Of course, if it is not desired to mount the camera on the side, the X-ray source may be moved and aligned with the side of the patient first to acquire the thickness of the patient, and then the X-ray source is moved and aligned with the detector, which only adds one more step to the entire process. Alternatively, in the process of positioning, the patient may be first allowed to stand on his/her side, and after a thickness value is acquired by the camera, the patient is allowed to stand facing or facing away for formal exposure. Of course, the method for acquiring the thickness of the present application does not only involve the distance between the front chest and the back. When the spine of the patient needs to be measured, the patient may also stand on his/her side, and the thickness of the patient in this case refers to the distance between the two sides of the patient. The method for acquiring a thickness of a patient may also be applicable to the above-described method.

According to the method for acquiring a thickness of a patient and the X-ray imaging system in some embodiments of the present invention, the target region including the region of interest of the patient is obtained by dividing the point cloud obtained by means of the optical image and the depth image acquired by the camera, and then the thickness of the patient may be calculated on the basis of two methods. The first method is to divide the target region into one or a plurality of key regions on the basis of the captured site and then calculate an overall thickness value of the patient on the basis of a thickness value of each key region. The process of acquiring a thickness value in this method is relatively simple, and the required calculation amount is not large, but this calculation method is limited by the distance between the patient and the detector. When the distance between the patient and the detector is large, the accuracy of the calculated thickness value will decrease. The second method is to fit two surfaces (e.g., the front chest and the back) of the patient on the basis of the side view or the top view of the point cloud. A thickness value of the patient acquired by this method is more accurate.

An exemplary embodiment of the present invention further provides a method for acquiring a thickness of a patient, including acquiring optical image data and depth image data captured by a camera; generating a point cloud on the basis of the optical image data and the depth image data, where the point cloud can be displayed in one or a plurality of views; extracting a target region in the point cloud, the target region including a region of interest of the patient; and acquiring a thickness value of the patient on the basis of at least one key region divided within the target region or on the basis of identifying a first surface and a second surface of the patient.

An exemplary embodiment of the present invention further provides an X-ray imaging method, including: acquiring optical image data and depth image data captured by a camera; generating a point cloud on the basis of the optical image data and the depth image data, where the point cloud can be displayed in one or a plurality of views; extracting a target region in the point cloud, the target region including a region of interest of the patient; acquiring a thickness value of the patient on the basis of at least one key region divided within the target region or on the basis of identifying a first surface and a second surface of the patient; and selecting exposure parameters on the basis of the thickness value to carry out exposure on the patient.

An exemplary embodiment of the present invention further provides an X-ray imaging system, including a control unit capable of executing the method for acquiring a thickness of a patient.

An exemplary embodiment of the present invention further provides an X-ray imaging system, including an apparatus for acquiring a thickness of a patient. The apparatus for acquiring a thickness of a patient includes an acquisition module, a generation module, an extraction module, and a calculation module. The acquisition module is configured to acquire optical image data and depth image data captured by a camera. The generation module is configured to generate a point cloud on the basis of the optical image data and the depth image data, where the point cloud can be displayed in one or a plurality of views. The extraction module is configured to extract a target region in the point cloud. The target region includes a region of interest of the patient. The calculation module is configured to acquire a thickness value of the patient on the basis of at least one key region divided within the target region or on the basis of identifying a first surface and a second surface of the patient.

The present invention may further provide a non-transitory computer-readable storage medium for storing an instruction set and/or a computer program. When executed by a computer, the instruction set and/or computer program causes the computer to execute the image processing distribution method. The computer executing the instruction set and/or computer program may be a computer of a medical imaging system, or may be other apparatuses/modules of the medical imaging system. In one embodiment, the instruction set and/or computer program may be programmed into a processor/control apparatus of the computer.

Specifically, when executed by the computer, the instruction set and/or computer program causes the computer to:
acquire optical image data and depth image data captured by a camera;
generate a point cloud on the basis of the optical image data and the depth image data, where the point cloud can be displayed in one or a plurality of views;
extract a target region in the point cloud, the target region including a region of interest of the patient; and
acquire a thickness value of the patient on the basis of at least one key region divided within the target region or on the basis of a first surface and a second surface of the patient that are fitted.

The instructions described above may be combined into one instruction for execution, and any of the instructions may also be split into a plurality of instructions for execution. Moreover, the present invention is not limited to the instruction execution order described above.

As used herein, the term “computer” may include any processor-based or microprocessor-based system, including a system using a microcontrol apparatus, a reduced instruction set computer (RISC), an application-specific integrated circuit (ASIC), a logic circuit, and any other circuit or processor capable of executing the functions described herein. The examples above are exemplary only and are not intended to limit the definition and/or meaning of the term “computer” in any way.

The instruction set may include various commands used to instruct the computer serving as a processing machine or the processor to perform specific operations, for example, methods and processes of various embodiments. The instruction set may be in the form of a software program that may form part of one or more tangible, non-transitory computer-readable media. The software may be in various forms of, for example, system software or application software. Furthermore, the software may be in the form of a standalone program or a collection of modules, a program module within a larger program, or part of a program module. The software may also include modular programming in the form of object-oriented programming. Processing of input data by the processing machine may be in response to an operator command, or in response to a previous processing result, or in response to a request made by another processing machine.

As used herein, the term “computer” may include any processor-based or microprocessor-based system, including a system using a microcontrol apparatus, a reduced instruction set computer (RISC), an application-specific integrated circuit (ASIC), a logic circuit, and any other circuit or processor capable of executing the functions described herein. The examples above are exemplary only and are not intended to limit the definition and/or meaning of the term “computer” in any way.

Some exemplary embodiments have been described above. However, it should be understood that various modifications may be made. For example, if the described technologies are executed in different orders and/or if assemblies in the described systems, architectures, devices or circuits are combined in different ways and/or replaced or supplemented by additional assemblies or equivalents thereof, suitable results can be achieved. Accordingly, other embodiments also fall within the scope of protection of the claims.

1. A method for acquiring a thickness of a patient, comprising:
acquiring optical image data and depth image data captured by a camera;
generating a point cloud on the basis of the optical image data and the depth image data, wherein the point cloud can be displayed in one or a plurality of views;
extracting a target region in the point cloud, the target region comprising a region of interest of the patient; and
acquiring a thickness value of the patient on the basis of a thickness value of at least one key region divided within the target region, or acquiring a thickness value of the patient on the basis of a distance between a first surface and a second surface of the patient that are fitted.

2. The method for acquiring a thickness of a patient according to claim 1, wherein the generating a point cloud on the basis of the optical image data and the depth image data comprises performing registration on the basis of the optical image data and the depth image data and generating an initial point cloud, and post-processing the initial point cloud to obtain the point cloud.

3. The method for acquiring a thickness of a patient according to claim 2, wherein the post-processing comprises hole filling and downsampling.

4. The method for acquiring a thickness of a patient according to claim 1, wherein the extracting a target region in the point cloud comprises: removing a background part in the point cloud to obtain the target region comprising the region of interest of the patient.

5. The method for acquiring a thickness of a patient according to claim 4, wherein the removing a background part in the point cloud comprises:
identifying a classification map corresponding to the point cloud on the basis of a classification learning model, and extracting the target region on the basis of the classification map; or
removing the background part on the basis of a source image distance (SID).

6. The method for acquiring a thickness of a patient according to claim 1, wherein the acquiring a thickness value of the patient on the basis of a thickness of one or a plurality of key regions divided within the target region comprises:
dividing the target region into at least one key region on the basis of a captured site of the patient; and
calculating an average value of a thickness value in the at least one key region respectively, calculating, when the target region is divided into a plurality of key regions, a thickness value of the patient in each key region respectively, and calculating an overall thickness value of the patient on the basis of a weight of each key region.

7. The method for acquiring a thickness of a patient according to claim 1, wherein the acquiring a thickness value of the patient on the basis of a distance between a first surface and a second surface of the patient that are fitted comprises:
on the basis of the thickness value in the target region, identifying the first surface of the patient that faces away from a detector;
fitting and identifying the second surface of the patient that is close to the detector; and
acquiring the thickness value of the patient on the basis of the distance between the first surface and the second surface.

8. The method for acquiring a thickness of a patient according to claim 7, wherein the fitting and identifying the second surface of the patient comprises:
fitting a plurality of planes of the patient that are close to the detector, the plurality of planes comprising a surface of the detector; and
identifying the second surface in the plurality of planes on the basis of the continuity of a histogram of the target region.

9. The method for acquiring a thickness of a patient according to claim 7, wherein the identifying the first surface of the patient that faces away from a detector comprises: determining a position at which a greatest value of a thickness in the target region is located, and defining a surface on which the position is located as the first surface of the patient.

10. An X-ray imaging method, comprising:
acquiring optical image data and depth image data captured by a camera;
generating a point cloud on the basis of the optical image data and the depth image data, wherein the point cloud can be displayed in one or a plurality of views;
extracting a target region in the point cloud, the target region comprising a region of interest of the patient;
acquiring a thickness value of the patient on the basis of a thickness of at least one key region divided within the target region, or acquiring a thickness value of the patient on the basis of a distance between a first surface and a second surface of the patient that are fitted; and
selecting exposure parameters on the basis of the thickness value to carry out exposure on the patient.

11. An X-ray imaging system, comprising an apparatus for acquiring a thickness of a patient, said apparatus comprising:
an acquisition module, configured to acquire optical image data and depth image data captured by a camera;
a generation module, configured to generate a point cloud on the basis of the optical image data and the depth image data, wherein the point cloud can be displayed in one or a plurality of views;
an extraction module, configured to extract a target region in the point cloud, the target region comprising a region of interest of the patient; and
a calculation module, configured to acquire a thickness value of the patient on the basis of a thickness of at least one key region divided within the target region, or acquire a thickness value of the patient on the basis of identifying a distance between a first surface and a second surface of the patient.
